(19) United States
(12) Patent Application Publication
(10) Pub. No.: US 2020/0381088 A1
(43) Pub. Date: Dec. 3, 2020

(54) METHOD AND SYSTEM FOR CLINICAL TRIAL RESOURCE MANAGEMENT USING BLOCK CHAIN

(71) Applicant: INDUSTRIAL COOPERATION FOUNDATION CHONBUK NATIONAL UNIVERSITY, Jeonju-si (KR)

(72) Inventors: Min Gul KIM, Jeonju-si (KR); Ji Hyoung LEE, Gwangju (KR); Sang Un JEONG, Jeonju-si (KR)

(73) Assignee: INDUSTRIAL COOPERATION FOUNDATION CHONBUK NATIONAL UNIVERSITY, Jeonju-si (KR)

(21) Appl. No.: 16/960,369

(22) PCT Filed: Jan. 8, 2019
(86) PCT No.: PCT/KR2019/000250

§ 371 Date: Jul. 7, 2020

(30) Foreign Application Priority Data
Jan. 12, 2018 (KR) 10-2018-0004385

(51) Int. Cl. G16H 10/20; H04L 9/08; G06Q 50/18; G06Q 30/00; G16H 20/10; G16H 10/60; G06Q 50/26; G16H 40/20

(52) U.S. Cl. CPC G16H 10/20; H04L 9/083; G06Q 50/18; G06Q 30/0185; G06Q 2220/00; G16H 10/60; G06Q 50/265; G16H 40/20; H04L 2209/38; G16H 20/10

(57) ABSTRACT

A method is disclosed including: allowing a contract research organization device to generate clinical trial information and transmit the information to a blockchain server; allowing the server to register the information, and assign a first private key and a first public key to a set site device and transmit the assigned keys to the site device in response to a request from the organization device; allowing the server to complete applicant authentication using a second private key assigned to an applicant for a clinical trial; allowing the site device to confirm previous participation information of the applicant; and allowing the site device to perform the clinical trial based on the previous participation information, encrypt a performance result of the clinical trial using the first public key, a second public key corresponding to the second private key, and the second private key, and transmit the encrypted performance result to the server.

### FIELD OF THE DISCLOSURE

The present invention relates to a method and system for clinical trial resource management using a blockchain, and more particularly, to a blockchain-based clinical-trial resource management method and system in which clinical trial records of subjects are managed based on a blockchain.

### DESCRIPTION OF RELATED ART

Clinical trials are experiments to check the stability and effectiveness of drugs in a human body when developing medicines for the treatment or prevention of diseases. In Phase 1, subjects are not allowed to participate in other clinical trials for three months after clinical trials in which they participated were completed in accordance with regulations about approval of clinical trial plans for medicines which are defined by the Ministry of Food and Drug Safety, formerly known as the Korea Food & Drug Administration.

Therefore, an execution organization which conducts a clinical trial checks whether subjects have participated in other clinical trials within two weeks from the starting day of a clinical trial through the Ministry of Food and Drug Safety. However, information provided by the Ministry of Food and Drug Safety just indicates whether an applicant has participated in a clinical trial but does not include information about the last drug administered during the clinical trial. Also, it is related to protect the privacy of subjects.

### SUMMARY OF THE INVENTION

Various embodiments of the present invention for solving such a conventional problem are directed to providing a blockchain-based clinical-trial resource management method and system that manage clinical trial records of subjects using a blockchain.

According to an aspect of the present invention, there is provided a method for clinical trial resource management using a blockchain, the method including causing a contract research organization device to generate clinical trial information and transmit the clinical trial information to a blockchain server, causing the blockchain server to register the clinical trial information and assign a first private key and a first public key to a set site device and transmit the assigned keys to the site device in response to a request from the contract research organization device, causing the blockchain server to complete applicant authentication using a second private key assigned to an applicant for a clinical trial, causing the site device to check participation history information of the applicant, which has been received from the blockchain server, to check whether the applicant can participate in the clinical trial when a consent form is filled out by the authenticated applicant, and causing the site device to perform the clinical trial on the basis of the participation history information of the clinical trial, encrypt a result of performing the clinical trial with the first public key, a second public key corresponding to the second private key, and the second private key, and transmit the encrypted performance result to the blockchain server.

Also, the causing of the site device to check participation history information of the applicant, which has been received from the blockchain server, to check whether the applicant can participate in the clinical trial when a consent form is filled out by the authenticated applicant may include causing the blockchain server to decrypt the participation history information, which is encrypted with the second private key, by using the second public key, and to check the decrypted participation history information, causing the site device to check whether the applicant is contracted to participate in another clinical trial on the basis of the participation history information, and causing the site device to check a date on which a drug was last administered for the applicant having participated in a previous clinical trial when the applicant is not contracted to another clinical trial.

Also, the method may further include causing the site device to make a clinical trial contract with the applicant when a difference between the current date and the last administration date is greater than or equal to a threshold value.

Also, the transmitting of the second private key assigned to the applicant for the clinical trial to the blockchain server may include causing the site device to receive login information including biometric information from the applicant, causing the site device to request the blockchain server to assign the second private key and the second public key when the second private key is not mapped to the login information, and causing the site device to receive the second private key and the second public key from the blockchain server and map the second private key to the login information.

Also, the method may further include causing the site device to encrypt personal information included in the consent form with the first public key, the second public key, and the second private key when the consent form is filled out and to transmit the encrypted personal information to the blockchain server.

According to another aspect of the present invention, there is provided a system for clinical trial resource management using a blockchain, the system including a contract research organization device configured to generate clinical trial information, a blockchain server configured to register the clinical trial information and assign a first private key and a first public key to a set site device in response to a request from the contract research organization device, and an site device configured to, when a consent form is filled out by an applicant for a clinical trial who is authenticated in the blockchain server using a second private key assigned to the applicant, check participation history information of the applicant received from the blockchain server so as to check whether the applicant can participate in the clinical trial, perform the clinical trial according to whether the applicant can participate, encrypt a result of performing the clinical trial with the first public key, a second public key corresponding to the second private key, and the second private key.

Also, the participation history information may be obtained by the blockchain server decrypting the participation history information of the applicant encrypted with the second private key using the second public key.

Also, the site device may check whether the applicant is contracted to participate in another clinical trial and a date on which a drug was last administered to the applicant when the applicant participated in a previous clinical trial on the basis of the participation history information, may make a clinical trial contract with the applicant when a difference between the current date and the last administration date is greater than or equal to a threshold value, and may encrypt personal information included in the consent form with the first public key, the second public key, and the second private key when the consent form is filled out.

Also, the site device may request the blockchain server to assign the second private key and the second public key when the second private key is not mapped to login information including biometric information received from the applicant and may map the second private key, which is one of the second private key and the second public key assigned by the blockchain server, to the login information according to the request.

As described above, the blockchain-based clinical trial resource management method and system of the present invention may manage clinical trial records of subjects using a blockchain, and thus it is possible to check whether the subjects have participated in clinical trials, check a date on which a drug was last administrated, and improve the security of the clinical trial records.

### BRIEF DESCRIPTION OF THE DRAWINGS

FIG. 1 is a diagram showing a clinical trial resource management system according to an embodiment of the present invention.

FIG. 2 is a flowchart illustrating a registration method for the purpose of clinical trial resource management according to an embodiment of the present invention.

FIG. 3 is a flowchart illustrating a method of performing pre-screening for the purpose of clinical trial resource management according to an embodiment of the present invention.

FIG. 4 is a flowchart illustrating a method of storing a clinical trial result for the purpose of clinical trial resource management according to an embodiment of the present invention.

FIG. 5 is a flowchart illustrating a method of performing pre-screening for the purpose of clinical trial resource management according to another embodiment of the present invention.

### DETAILED DESCRIPTION OF THE INVENTION

Hereinafter, exemplary embodiments of the present invention will be described in more detail with reference to the accompanying drawings. At this time, it should be noted that like reference numerals denote like elements in the accompanying drawings. Also, a detailed description of well-known functions or configurations will be omitted in order not to unnecessarily obscure the subject matter of the present invention.

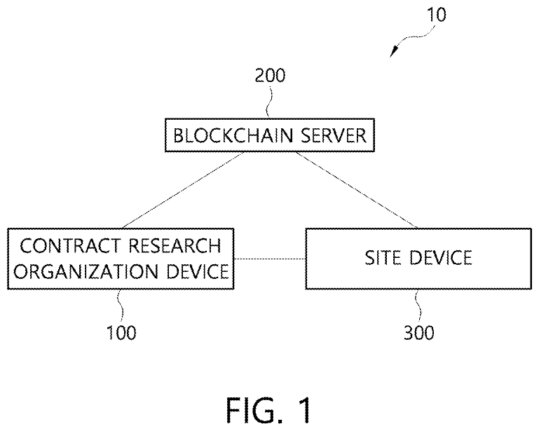

FIG. 1 is a diagram showing a clinical trial resource management system according to an embodiment of the present invention.

Referring to FIG. 1, a clinical trial resource management system 10 according to the present invention includes a contract research organization device 100, a blockchain server 200, and an site device 300.

The contract research organization device 100 is a device which is used for a contract research organization (CRO) and may be an electronic device such as a computer. Also, an application running based on a blockchain network to entrust a clinical trial may be installed in the contract research organization device 100. Also, the contract research organization device 100 generates clinical trial information by a user who manages the contract research organization device 100 through an application, and sets an execution organization to perform a clinical trial. The contract research organization device 100 transmits the clinical trial information and the execution organization information to the blockchain server 200. To this end, the contract research organization device 100 may communicate with the blockchain server 200 through wireless communication such as Fifth-Generation mobile communications (5G), Long Term Evolution-Advanced (LTE-A), Long Term Evolution (LTE), and Wireless Fidelity (WiFi) or wired communication using a cable. Here, the clinical trial information may be detailed information about a clinical trial, which includes the name of the clinical trial, subjects for the clinical trial, the start date and the end date of the clinical trial, a physical examination schedule for conducting the clinical trial, and a drug administration schedule. In addition, the execution organization information may include the name, location, and the like of the execution organization.

The blockchain server 200 registers the clinical trial on the basis of the clinical trial information received from the contract research organization device 100 and assigns a first security key to the site device 300 on the basis of the execution organization information received from the contract research organization device 100. The blockchain server 200 assigns a second security key to an applicant who intends to access the blockchain server 200 through the site device 300. In this case, the first security key is formed to pair a first public key with a first private key, and the second security key is formed to pair a second public key with a second private key. The blockchain server 200 stores participation information and personal information which are for a plurality of applicants and which are received from a plurality of site device. In this case, it is preferable that the participation information and the personal information stored in the blockchain server 200 be encrypted.

The site device 300 is a device which is used in hospitals, research institutes, and the like for conducting clinical trials and may be an electronic device such as a computer. Also, an application running based on a blockchain network may be installed in the site device 300. Through communication with the blockchain server 200, the site device 300 performs pre-screening for checking whether an applicant who applies for a clinical trial has participated in another clinical trial, stores the clinical trial result of an applicant, etc. To this end, the site device 300 may communicate with the blockchain server 200 through wireless communication such as Fifth-Generation mobile communications (5G), Long Term Evolution-Advanced (LTE-A), Long Term Evolution (LTE), and Wireless Fidelity (WiFi) or wired communication using a cable.

In detail, when an execution organization where the site device 300 is located is set by the contract research organization device 100, the site device 300 receives the assigned first security key and the clinical trial information from the blockchain server 200 and then stores the received first security key and clinical trial information. The site device 300 receives applicant login information from an applicant who intends to participate in the clinical trial. To this end, the applicant may visit the execution organization where the site device 300 is located and then enter the login information through the site device 300. The site device 300 receives the login information including biometric information from the applicant. The biometric information may be information including iris information, facial recognition information, fingerprint information, and like of the applicant.

When it is determined that the second private key, which is one of the second public key and the second private key included in the second security key, is mapped to the login information, the site device 300 transmits the second private key mapped to the login information to the blockchain server 200. Subsequently, when the applicant authentication is completed through the second private key in the blockchain server 200, the site device 300 establishes a channel with the blockchain server 200. Here, the channel may be a channel associated with the applicant, and the blockchain server 200 may grant access to the applicant from the site device 300 through the channel establishment.

When it is determined that the second security key is not mapped to the login information, the site device 300 transmits a request signal for assigning the second security key to the blockchain server 200. The site device 300 receives the assigned second security key from the blockchain server 200 according to the request signal and maps the second private key included in the second security key to the login information. The site device 300 transmits the second private key mapped to the login information to the blockchain server 200, and a channel is established between the blockchain server 200 and the site device 300.

When the channel is established in this way, the site device 300 performs pre-screening on the applicant. There may be various methods for the pre-screening, but in the description of the present invention, two of the methods will be described.

According to an embodiment, the site device 300 receives information necessary to fill out the consent form from the applicant. When the consent form is filled out, the site device 300 receives participation history information of the applicant from the blockchain server 200 in order to perform pre-screening. In detail, since the blockchain server 200 establishes a channel with the site device 300 in order to grant access to the applicant, the blockchain server 200 may determine that the applicant is a person who has accessed the blockchain server 200. Accordingly, the blockchain server 200 may check the participation history information of the applicant. Here, the participation history information may include whether a contract made with another execution organization by the applicant has been terminated, whether the applicant has participated in another clinical trial, a date on which a drug was last administered in another clinical trial, and the like. Also, since the participation history information is encrypted with the second private key mapped to the login information of the applicant and is stored in the blockchain server 200, the blockchain server 200 may decrypt the participation history information using the second public key.

The site device 300 checks whether it is possible to make a contract with the applicant on the basis of the participation history information received from the blockchain server 200. When the participation history information indicates that the contract made with another execution organization by the applicant has not been terminated, the site device 300 determines that no contract is possible and displays an associated message. When the contract made with another execution organization by the applicant has been terminated, the site device 300 determines that a contract is possible and performs pre-screening on the applicant. In this case, when it is determined that the applicant has never made a contract with another execution organization, the site device 300 determines that the applicant has never participated in a clinical trial and does not perform pre-screening.

The site device 300 determines the date on which the drug was last administered to the applicant in another clinical trial on the basis of the participation history information received from the blockchain server 200. When 90 days have elapsed from the date on which the drug was last administered, the site device 300 determines that the applicant can participate in the clinical trial and makes the contract with the applicant.

The site device 300 encrypts personal information written in the filled-out consent form and transmits the encrypted personal information to the blockchain server 200. Here, the personal information may include applicant information, execution organization information, a date on which the consent form is filled out, details written on the consent form, and the like. The site device 300 applies a hash function to the personal information to generate a signature and encrypts the generated signature and the personal information with the second private key. The site device 300 re-encrypts the information encrypted with the second private key and the second public key using the first public key. As described above, by using both of the first security key assigned to the site device 300 and the second security key assigned to the applicant when the personal information is encrypted, it is possible to enhance the security of the personal information, and also it is possible to guarantee the integrity of the personal information. That is, the personal information of the applicant may be checked by the site device 300 only upon request of the applicant.

According to another embodiment, the site device 300 receives information necessary to fill out the consent form from the applicant. When the consent form is filled out, the site device 300 encrypts the personal information included in the consent form. The site device 300 transmits the encrypted personal information to the blockchain server 200. Here, the personal information may include applicant information, execution organization information, a date on which the consent form is filled out, details written on the consent form, the second private key, and the like. The site device 300 applies a hash function to the personal information to generate a signature and encrypts the generated signature and the personal information with the second private key. The site device 300 re-encrypts the information encrypted with the second private key and the second public key using the first public key.

The site device 300 receives the participation history information for the applicant from the blockchain server 200 and performs pre-screening. In detail, the blockchain server 200 may check the participation history information of the applicant. Here, the participation history information may include whether a contract made with another execution organization by the applicant has been terminated, whether the applicant has participated in another clinical trial, a date on which a drug was last administered in another clinical trial, and the like. Also, since the participation history information is encrypted with the second private key mapped to the login information of the applicant and is stored in the blockchain server 200, the blockchain server 200 may decrypt the participation history information using the second public key and check the decrypted participation history information.

The site device 300 checks whether it is possible to make a contract with the applicant on the basis of the participation history information received from the blockchain server 200. When the participation history information indicates that the contract made with another execution organization by the applicant has not been terminated, the site device 300 determines that no contract is possible and displays an associated message. When the contract made with another execution organization by the applicant has been terminated, the site device 300 determines that a contract is possible and performs pre-screening. In this case, when it is determined that the applicant has never made a contract with another execution organization, the site device 300 determines that the applicant has never participated in a clinical trial and does not perform pre-screening.

The site device 300 determines the date on which the drug was last administered to the applicant in another clinical trial on the basis of the participation history information received from the blockchain server 200. When 90 days have elapsed from the date on which the drug was last administered, the site device 300 determines that the applicant can participate in the clinical trial and makes the contract with the applicant.

When the contract is made with the applicant, the site device 300 checks whether a clinical trial event has arrived according to the stored clinical trial information. Here, the clinical trial event refers to an event corresponding to a schedule to be conducted during the clinical trial, such as a physical examination schedule, a drug administration schedule, and the like. When the clinical trial event has arrived, the site device 300 outputs a notification indicating the arrival of the event and performs the clinical trial event.

The site device 300 inputs a performance result, which is a result of performing the clinical trial event. In detail, the site device 300 decrypts the encrypted personal information after the consent form is filled out. In this case, the site device 300 performs the decryption using the first private key assigned to the site device 300. When the personal information is decrypted with the first private key, the second public key and the information encrypted with the second private key are derived. The site device 300 may use the second public key to decrypt the information encrypted with the second private key. When the information encrypted with the second private key is decrypted using the second public key, the personal information and the signature generated by applying the hash function to the personal information are derived. The site device 300 may generate the signature by applying the hash function to the derived personal information. When the generated hash is the same as the signature derived through the decryption, the site device 300 may determine that the integrity of the personal information is guaranteed.

The site device 300 updates the personal information using the performance result obtained by performing the clinical trial and re-encrypts the updated personal information. The site device 300 transmits the re-encrypted personal information to the blockchain server 200. Here, the performance result may include physical conditions of the applicant (height, weight, blood sugar, blood pressure, etc.) before the participation in the clinical trial, physical conditions of the applicant after the participation in the clinical trial, and the type of the drug administered to the applicant when participating in the clinical trial, the number and date of administrations, and the like.

When the clinical trial is terminated or when a signal for terminating the contract is received from the applicant, the site device 300 terminates the contract with the applicant. The site device 300 encrypts participation information of the applicant using the second public key. Here, the participation information may include whether a contract made with an execution organization by the applicant has been terminated, whether the applicant has participated in a clinical trial, a date on which a drug was last administered in a clinical trial, and the like. The site device 300 transmits the encrypted participation information to the blockchain server 200. Accordingly, when other execution organizations intend to check the participation information of the applicant, the execution organizations can check only information regarding whether the contract made with the execution organization by the applicant has been terminated, whether the applicant has participated in the clinical trial, and the date on which the drug was last administered during the clinical trial and cannot check the type of the clinical trial in which the applicant has participated, the result of the clinical trial, and the like. Therefore, it is possible to maintain the security of the personal information of the applicant.

In addition, in the present invention, it is described as an example that the applicant accesses the blockchain server 200 through the site device 300. However, the present invention is not limited thereto, and the applicant may access the blockchain server 200 through the site device 300 using a personal terminal (not shown) assigned to the applicant, such as a cellular phone, a computer, a tablet PC, and the like. In this case, the personal terminal and the site device 300 may be connected to each other through wired and/or wireless communication.

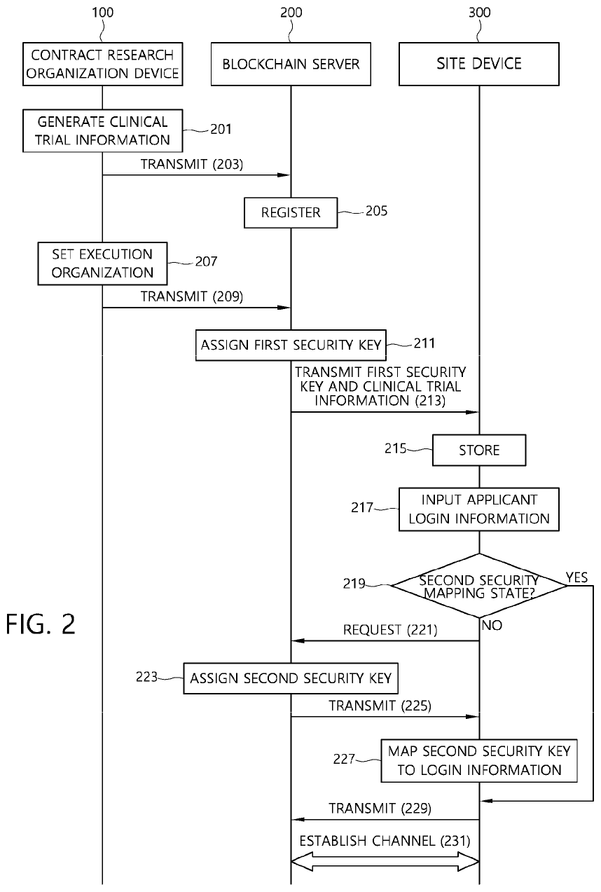

FIG. 2 is a flowchart illustrating a registration method for the purpose of clinical trial resource management according to an embodiment of the present invention.

Referring to FIG. 2, in operation 201, the contract research organization device 100 generates clinical trial information for a clinical trial to be conducted. Here, the clinical trial information may be detailed information about a clinical trial, which includes the name of the clinical trial, subjects for the clinical trial, the start date and the end date of the clinical trial, a physical examination schedule for conducting the clinical trial, and a drug administration schedule. In operation 203, the contract research organization device 100 transmits the clinical test information to the blockchain server 200. In operation 205, the blockchain server 200 registers the clinical trial information.

In operation 207, the contract research organization device 100 sets an execution organization to conduct the clinical trial. In operation 209, the contract research organization device 100 transmits information regarding the set execution organization to the blockchain server 200. Here, the information regarding the execution organization may include the name, location, and the like of the execution organization. In operation 211, the blockchain server 200 assigns a first security key to a device to be used in the set execution organization. The first security key is a security key to be used upon data authentication through the blockchain server 200 and is generated as a pair of a first public key and a first private key. In operation 213, the blockchain server 200 transmits the assigned first security key and the clinical trial information to the site device 300. Here, the site device 300 is a device which is used in the execution organization to conduct the clinical trial, for example, in hospitals, research institutes, and the like and may be an electronic device such as a computer. In operation 215, the site device 300 stores the first security key.

In operation 217, the site device 300 receives applicant login information from an applicant who intends to participate in the clinical trial. To this end, the site device 300 may be lent to the applicant by an administrator who manages the site device 300 so that the applicant can enter the login information. The site device 300 receives the login information including biometric information from the applicant. The biometric information may be information including iris information, facial recognition information, fingerprint information, and like of the applicant. In operation 219, when it is determined that a second private key, which is included in a second security key including the second private key and a second public key, is mapped to the login information, the site device 300 performs operation 229. In operation 229, the site device 300 transmits the second private key mapped to the login information to the blockchain server 200. In operation 231, the blockchain server 200 performs applicant authentication and establishes a channel with the site device 300. Here, the channel may be a channel associated with the applicant, and the blockchain server 200 may grant access to the applicant from the site device 300 through the channel establishment.

In operation 219, when it is determined that the second security is not mapped to the login information, the site device 300 performs operation 221. In operation 221, the site device 300 transmits a request signal for assigning the second security key to the blockchain server 200. In operation 223, the blockchain server 200 may assign the second security key to the applicant. In operation 225, the blockchain server 200 may transmit the assigned second security key to the site device 300. The site device 300 maps the second private key included in the second security key to the login information in operation 227 and then performs operation 229. In operation 229, the site device 300 transmits the second private key mapped to the login information to the blockchain server 200. In operation 231, the blockchain server 200 establishes a channel with the site device 300.

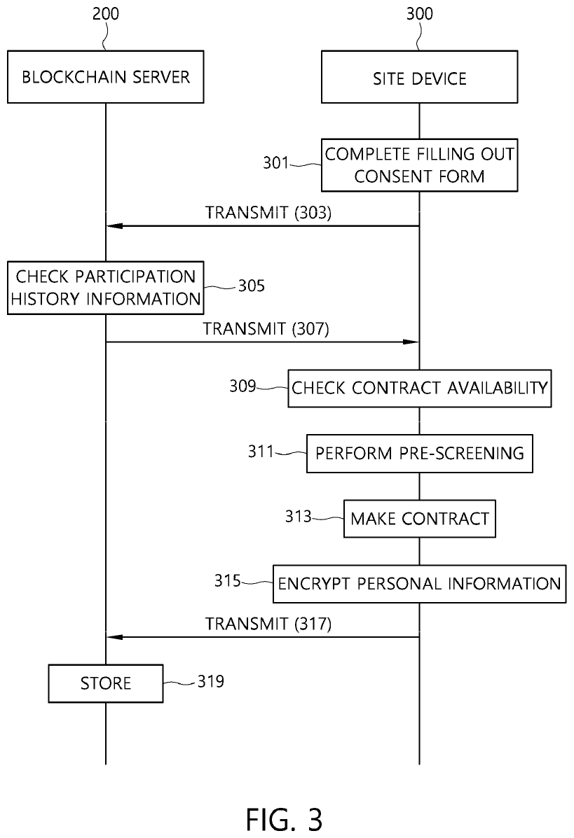

FIG. 3 is a flowchart illustrating a method of performing pre-screening for the purpose of clinical trial resource management according to an embodiment of the present invention.

Referring to FIGS. 2 and 3, when a channel is established between the blockchain server 200 and the site device 300 as shown in FIG. 2, the site device 300 performs operation 301. In operation 301, the site device 300 receives information necessary to fill out the consent form from the applicant. When the consent form is filled out, the site device 300 performs operation 303. In operation 303, since the consent form is filled out, the site device 300 transmits an information request signal for performing pre-screening to the blockchain server 200. In operation 305, the blockchain server 200 checks participation history information of the applicant. In operation 307, the blockchain server 200 transmits the participation history information to the site device 300.

In detail, since the blockchain server 200 establishes a channel with the site device 300 in order to grant access to the applicant in operation 231 of FIG. 2, the blockchain server 200 may determine that the applicant is a person who has accessed the blockchain server 200. Accordingly, the blockchain server 200 may check the participation history information of the applicant. Here, the participation history information may include whether a contract made with another execution organization by the applicant has been terminated, whether the applicant has participated in another clinical trial, a date on which a drug was last administered in another clinical trial, and the like. Also, since the participation history information is encrypted with the second private key mapped to the login information of the applicant and is stored in the blockchain server 200, the blockchain server 200 may decrypt the participation history information using the second public key and check the decrypted participation history information.

In operation 309, the site device 300 checks whether it is possible to make a contract with the applicant on the basis of the participation history information received from the blockchain server 200. When the participation history information indicates that the contract made with another execution organization by the applicant has not been terminated, the site device 300 determines that no contract is possible and displays an associated message. When the contract made with another execution organization has been terminated, the site device 300 determines that a contract is possible and performs operation 311.

In operation 311, the site device 300 performs pre-screening. The site device 300 checks the date on which the drug was last administered to the applicant in another clinical trial on the basis of the participation history information received from the blockchain server 200. When 90 days have elapsed from the date on which the drug was last administered, the site device 300 determines that the applicant can participate in the clinical trial and then performs operation 313. The site device 300 makes a contract with the applicant in operation 313 and then performs operation 315.

In operation 315, the site device 300 encrypts the personal information written in the consent form filled out in operation 301 and then performs operation 317. In operation 317, the site device 300 transmits the encrypted personal information to the blockchain server 200. In operation 319, the blockchain server 200 stores the encrypted personal information. Here, the personal information may include applicant information, execution organization information, a date on which the consent form is filled out, details written on the consent form, and the like. The site device 300 applies a hash function to the personal information to generate a signature and encrypts the generated signature and the personal information with the second private key. The site device 300 re-encrypts the information encrypted with the second private key and the second public key using the first public key. As described above, by using both of the first security key assigned to the site device and the second security key assigned to the applicant when the personal information is encrypted, it is possible to enhance the security of the personal information, and it is also possible to guarantee the integrity of the personal information. That is, the personal information of the applicant may be checked by the site device 300 only upon request of the applicant.

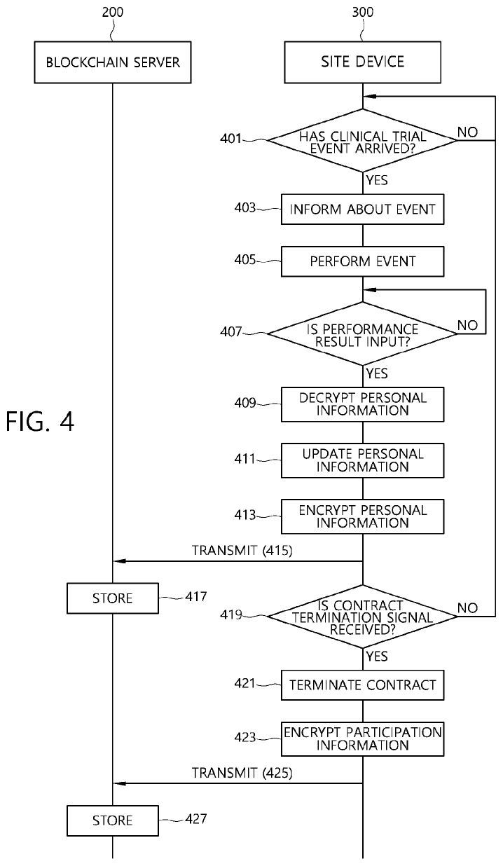

FIG. 4 is a flowchart illustrating a method of storing a clinical trial result for the purpose of clinical trial resource management according to an embodiment of the present invention.

Referring to FIGS. 2 to 4, when it is determined, according to the clinical trial information stored in operation 215 of FIG. 2, that a clinical trial event has arrived in operation 401, the site device 300 performs operation 403. When it is not determined that clinical trial event has arrived, the site device 300 continuously checks for the arrival. Here, the clinical trial event refers to an event corresponding to a schedule to be conducted during the clinical trial, such as a physical examination time, a drug administration time, and the like. The site device 300 outputs a notification indicating that the clinical trial event has arrived in operation 403 and then performs operation 405. The site device 300 performs the clinical trial event in operation 405 and then performs operation 407.

When a signal for inputting the performance result corresponding to the performed clinical trial event is input in operation 407, the site device 300 performs operation 409. In operation 409, the site device 300 decrypts the personal information encrypted in operation 315 of FIG. 3. In this case, the site device 300 performs the decryption using the first private key assigned to the site device 300. When the personal information is decrypted with the first private key, the second public key and the information encrypted with the second private key may be derived. The site device 300 may decrypt the information encrypted with the second private key using the second public key. When the site device 300 uses the second public key to decrypt the information encrypted with the second private key, the personal information and the signature generated by applying the hash function to the personal information are derived. The site device 300 may generate the signature by applying the hash function to the personal information. When the generated hash is the same as the signature derived through the decryption, the site device 300 may determine that the integrity of the personal information is guaranteed.

The site device 300 updates the personal information using the performance result obtained by performing the clinical trial in operation 411 and then performs operation 413. Here, the performance result for updating the personal information may include physical conditions of the applicant (height, weight, blood sugar, blood pressure, etc.) before the participation in the clinical trial, physical conditions of the applicant after the participation in the clinical trial, and the type of the drug administered to the applicant when participating in the clinical trial, the number and date of administrations, and the like. The site device 300 re-encrypts the updated personal information in operation 413 and then performs operation 415. In operation 415, the site device 300 transmits the encrypted personal information to the blockchain server 200. In operation 417, the blockchain server 200 stores the encrypted personal information.

When the clinical trial is terminated or when a signal for terminating the contract is received from the applicant in operation 419, the site device 300 performs operation 421. When the signal for terminating the contract is not received, the site device 300 returns to operation 401 and re-performs the above operation. The site device 300 terminates the contract with the applicant in operation 421 and then performs operation 423. In operation 423, the site device 300 encrypts participation information of the applicant using the second private key mapped to the login information of the applicant. Here, the participation information may include whether a contract made with an execution organization by the applicant has been terminated, whether the applicant has participated in a clinical trial, a date on which a drug was last administered in a clinical trial, and the like. In operation 425, the site device 300 may transmit the encrypted participation information to the blockchain server 200. In operation 427, the blockchain server 200 may store the participation information.

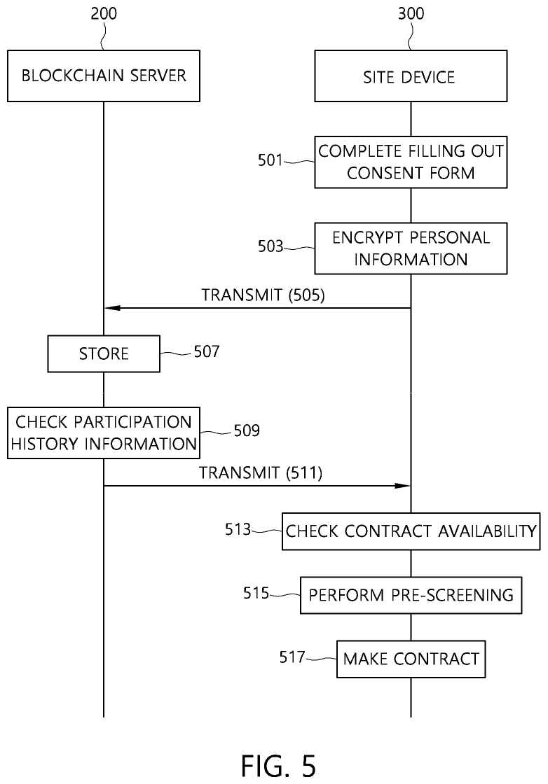

FIG. 5 is a flowchart illustrating a method of performing pre-screening for the purpose of clinical trial resource management according to another embodiment of the present invention.

Referring to FIGS. 2, 4, and 5, when a channel is established between the blockchain server 200 and the site device 300 as shown in FIG. 2, the site device 300 performs operation 501. In operation 501, the site device 300 receives information necessary to fill out the consent form from the applicant. When the consent form is filled out, the site device 300 performs operation 503. In operation 503, the site device 300 encrypts personal information. In operation 505, the site device 300 transmits the encrypted personal information to the blockchain server 200. In operation 507, the blockchain server 200 stores the encrypted personal information. In this case, the personal information may include applicant information, execution organization information, a date on which the consent form is filled out, details written on the consent form, the second private key, and the like. The site device 300 applies a hash function to the personal information to generate a signature and encrypts the generated signature and the personal information with the second private key. The site device 300 re-encrypts the information encrypted with the second private key and the second public key using the first public key. As described above, by using both of the first security key assigned to the site device and the second security key assigned to the applicant when the personal information is encrypted, it is possible to enhance the security of the personal information, and it is also possible to guarantee the integrity of the personal information. That is, the personal information of the applicant may be checked by the site device 300 only upon request of the applicant.

In operation 509, since the encrypted personal information is received from the site device 300, the blockchain server 200 checks participation history information of the applicant so that the site device 300 may perform pre-screening. In detail, since the blockchain server 200 establishes a channel with the site device 300 in order to grant access to the applicant in operation 231 of FIG. 2, the blockchain server 200 may determine that the applicant is a person who has accessed the blockchain server 200. Accordingly, the blockchain server 200 may check the participation history information of the applicant. Here, the participation history information may include whether a contract made with another execution organization by the applicant has been terminated, whether the applicant has participated in another clinical trial, a date on which a drug was last administered in another clinical trial, and the like. Also, since the participation history information is encrypted with the second private key mapped to the login information of the applicant and is stored in the blockchain server 200, the blockchain server 200 may decrypt the participation history information using the second public key and check the decrypted participation history information.

In operation 511, the blockchain server 200 transmits the checked participation history information to the site device 300. In operation 513, the site device 300 may check whether it is possible to make a contract with the applicant on the basis of the participation history information received from the blockchain server 200. When the participation history information indicates that the contract made with another execution organization by the applicant has not been terminated, the site device 300 determines that another contract is impossible and displays an associated message. When the contract made with another execution organization has been terminated, the site device 300 determines that a contract is possible and performs operation 515.

In operation 515, the site device 300 performs pre-screening. The site device 300 determines the date on which the drug was last administered to the applicant in another clinical trial on the basis of the participation history information received from the blockchain server 200. When 90 days have elapsed from the date on which the drug was last administered, the site device 300 determines that the applicant can participate in the clinical trial and then performs operation 517. In operation 517, the site device 300 makes a contract with the applicant.

As described above, when the contract is made with the applicant, the site device 300 may perform the clinical trial on the applicant. This has been described in detail with reference to FIG. 4, and thus a description thereof will be omitted.

The embodiments of the present invention disclosed in the present specification and the accompanying drawings have been provided only as specific examples in order to easily explain the technical content of the present invention and assist in understanding the present invention and do not limit the scope of the present invention. That is, it is obvious to those skilled in the art to which the present invention pertains that various modifications may be made without departing from the technical spirit of the present invention.

1. A method for clinical trial resource management, the method comprising:
causing a contract research organization device to generate clinical trial information and transmit the clinical trial information to a blockchain server;
causing the blockchain server to register the clinical trial information and assign a first private key and a first public key to a set site device and transmit the assigned keys to the site device in response to a request from the contract research organization device;
causing the blockchain server to complete applicant authentication using a second private key assigned to an applicant for a clinical trial;
causing the site device to check participation history information of the applicant, which has been received from the blockchain server, to check whether the applicant can participate in the clinical trial when a consent form is filled out by the authenticated applicant; and
causing the site device to perform the clinical trial on the basis of the participation history information of the clinical trial, encrypt a result of performing the clinical trial with the first public key, a second public key corresponding to the second private key, and the second private key, and transmit the encrypted performance result to the blockchain server.

2. The method of claim 1, wherein the causing of the site device to check participation history information of the applicant, which has been received from the blockchain server, to check whether the applicant can participate in the clinical trial when a consent form is filled out by the authenticated applicant comprises:
causing the blockchain server to decrypt the participation history information of the applicant, which is encrypted with the second private key, by using the second public key, and to check the decrypted participation history information;
causing the site device to check whether the applicant is contracted to participate in another clinical trial on the basis of the participation history information; and
causing the site device to check a date on which a drug was last administered to the applicant having participated in a previous clinical trial when the applicant is not contracted to another clinical trial.

3. The method of claim 2, further comprising causing the site device to make a clinical trial contract with the applicant when a difference between the current date and the last administration date is greater than or equal to a threshold value.

4. The method of claim 3, wherein the transmitting of the second private key assigned to the applicant for the clinical trial to the blockchain server comprises:
causing the site device to receive login information including biometric information from the applicant;
causing the site device to request the blockchain server to assign the second private key and the second public key when the second private key is not mapped to the login information; and
causing the site device to receive the second private key and the second public key from the blockchain server and map the second private key to the login information.

5. The method of claim 4, further comprising causing the site device to encrypt personal information included in the consent form with the first public key, the second public key, and the second private key when the consent form is filled out and to transmit the encrypted personal information to the blockchain server.

6. A system for clinical trial resource management, the system comprising:
a contract research organization device configured to generate clinical trial information;
a blockchain server configured to register the clinical trial information and assign a first private key and a first public key to a set site device in response to a request from the contract research organization device; and
an site device configured to, when a consent form is filled out by an applicant for a clinical trial who is authenticated in the blockchain server using a second private key assigned to the applicant, check participation history information of the applicant received from the blockchain server so as to check whether the applicant can participate in the clinical trial, perform the clinical trial according to whether the applicant can participate, encrypt a result of performing the clinical trial with the first public key, a second public key corresponding to the second private key, and the second private key.

7. The system of claim 6, wherein the participation history information is obtained by the blockchain server decrypting the participation history information of the applicant encrypted with the second private key using the second public key.

8. The system of claim 7, wherein the site device checks whether the applicant is contracted to participate in another clinical trial and a date on which a drug was last administered to the applicant when the applicant participated in a previous clinical trial on the basis of the participation history information, makes a clinical trial contract with the applicant when a difference between the current date and the last administration date is greater than or equal to a threshold value, and encrypts personal information included in the consent form with the first public key, the second public key, and the second private key when the consent form is filled out.

9. The system of claim 8, wherein the site device requests the blockchain server to assign the second private key and the second public key when the second private key is not mapped to login information including biometric information received from the applicant and maps the second private key, which is one of the second private key and the second public key assigned by the blockchain server, to the login information according to the request.
